(19) United States
(12) Patent Application Publication
(10) Pub. No.: US 2003/0148284 A1
(43) Pub. Date: Aug. 7, 2003

(54) Solid phase detection of nucleic acid molecules

(76) Inventors: Todd J. Vision, Carrboro, NC (US); Amber Carmon, Ithaca, NY (US); Theodore W. Thannhauser, Newfield, NY (US); Stephen Kresovich, Ithaca, NY (US); Sharon E. Mitchell, Ithaca, NY (US); Uwe R. Muller, Painted Post, NY (US)

Correspondence Address: Michael L. Goldman, Esq., NIXON PEABODY LLP, Clinton Square, P.O. Box 31051, Rochester, NY, 14603-1051, US

(21) Appl. No.: 10/023,337

(22) Filed: Dec. 17, 2001

(51) Int. Cl. C12Q 1/68; C12P 19/34

(52) U.S. Cl. 435/6; 435/91.2

(57) ABSTRACT

The present invention is directed to a method for detecting a target nucleic acid molecule in a sample. An immobilized oligonucleotide primer is extended using a polymerase, yielding an extension product that can be used in a detection assay. The assay is useful for detecting the presence of a target nucleic acid molecule in a sample and quantifying the amount of the target nucleic acid molecule in the sample. Also disclosed are ways of applying the method of the present invention.

### FIELD OF THE INVENTION

The present invention is directed to a method of solid phase detection of target nucleic acid molecules in samples.

### BACKGROUND OF THE INVENTION

Rapid, reliable, and sensitive nucleic acid detection assays are extremely important in the field of molecular biology and genetics. Nucleic acid detection assays can be used in a wide variety of applications, including, but not limited to: (1) pathogen detection; (2) disease diagnostics; (3) genotyping; and (4) expression studies. The usefulness of a nucleic acid detection assay is often tied to its efficiency, reliability, sensitivity, and cost-effectiveness. These characteristics are easily evident in the area of genotyping, which relies heavily on a high-throughput format in order to yield meaningful results in a cost-effective manner.

Due in part to the significant public interest in genomics research, there is a need for increasingly reliable and economic genotyping assays. An ideal genotyping assay would have a number of features: (1) it would be easily automatable; (2) it would be quantitative (e.g., it can measure the relative concentrations of different alleles in a sample); (3) it would discriminate between all non-identical alleles; (4) it would not require expensive equipment or expensive reagents; and (5) it would not require knowledge of the exact nature of differences among alleles in order to discriminate them from one another.

Although there are a number of effective nucleic acid detection assays currently available, many of them are tedious, costly, and time-consuming. Further, a large number of these assays require multiple handling steps, which can adversely affect the reliability of the results due to contamination problems. Frequently, low concentrations of the target nucleic acid molecule of interest contribute to the inability to detect the target nucleic acid molecule in the sample. The development of the polymerase chain reaction (“PCR”) as a method for amplifying nucleic acids in samples has revolutionized modern life sciences research, and has improved the ability to develop sensitive and reliable nucleic acid detection assays. The basic PCR method is described in detail in U.S. Pat. Nos. 4,683,195, 4,683,202, and 4,800,159. Over the years, numerous PCR-based techniques have been developed for a variety of applications. These assays have greatly enhanced the ability to amplify nucleic acids and to obtain direct sequence information from as little as one copy of a target nucleic acid sequence.

PCR is typically performed by placing a sample nucleic acids mixture in a thermocycler and subjecting the samples to three distinct temperature cycles, commonly referred to in the art as the denaturing, annealing, and synthesizing stages. The sample mixture typically comprises the target nucleic acid molecule (often comprising a double-stranded DNA molecule), a mixture of deoxynucleoside triphosphates, a pair of primers, a heat stable DNA polymerase (e.g., Taq polymerase), and a buffer solution. The primers are specific to and define the nucleic acid region targeted for amplification. In the denaturation stage, the temperature is raised to a temperature sufficient to separate the two strands of the DNA sample, resulting in single-stranded DNA templates for amplification. In the next stage of the cycle (i.e., the annealing stage), the temperature is lowered to allow for the generation of primed templates, during which stage the primers anneal to the single-stranded target templates. In the third stage of the cycle (i.e., the synthesizing stage), the temperature is raised to allow for binding of the DNA polymerase and for synthesis of the target nucleic acid. The cycle of strand separation, annealing of the primers, and synthesis of the target nucleic acid is repeated for about 20 to 60 cycles. The resulting nucleic acid molecule copies made in a given cycle serve as templates for the succeeding cycle. The number of target nucleic acid molecule copies increases approximately two-fold in each cycle. Although PCR is a powerful tool, recovery of amplification products may require the performance of tedious purification procedures, such as organic extraction, gel electrophoresis, centrifugation, and/or column purification (Maniatis et al., Molecular Cloning: A Laboratory Manual (1^{st} Edition) (Cold Spring Harbor Laboratory Press 1982)).

Modified microtiter wells are well known in the art as a means for capturing PCR products, commonly referred to in the art as “amplicons,” on a solid support (also referred to herein as a “solid substrate”) prior to hybridization (Kohsaka et al., “Microtiter Format Gene Quantification By Covalent Capture of Competitive PCR Products: Application to HIV-1 Detection,” Nucleic Acids Res. 21:3469-3472 (1993); Giorda et al., “Non-Radioisotopic Typing of Human Leukocyte Antigen Class II Genes On Microplates,” Biotechniques 15:918-925 (1993); Alard et al., “Charpentien B: A Versatile ELISA-PCR Assay for mRNA Quantification From a Few Cells,” Biotechniques 15:730-737 (1993)). For example, in one reported method, 5′-phosphorylated DNA primers are bound to secondary amines on microtiter well surfaces using standard carbodiimide condensation (Rasmussen et al., “Combined Polymerase Chain Reaction-Hybridization Microplate Assay Used to Detect Bovine Leukemia Virus and Salmonella,” Clin. Chem. 40:200-205 (1994); Oroskar et al., “Detection of Immobilized Amplicons by ELISA-Like Techniques,” Clin. Chem. 42:1547-1555 (1996)). However, many of the methods involving the capture of amplicons on solid supports such as microtiter wells still require the amplicons to be transferred from one well to another during the process, thereby causing problems due to contamination.

It is known in the art that combining the PCR amplification and immobilization stages into a single step is useful in decreasing the risk of contamination and in improving the efficiency of the amplification process (Kohsaka et al., “Solid-Phase Polymerase Chain Reaction,” J. Clin. Lab. Anal. 8:452-455 (1994); Andreadis et al., “Use of Immobilized PCR Primers to Generate Covalently Immobilized DNAs for In Vitro Transcription/Translation Reactions,” Nucleic Acids Res. 28(2):e5 (2000)). For example, Andreadis et al. have analyzed various covalent chemical attachment methods for immobilizing one of the PCR primers in a pair onto controlled pore glass (“CPG”) and/or polymer supports (Andreadis et al., “Use of Immobilized PCR Primers to Generate Covalently Immobilized DNAs for In Vitro Transcription/Translation Reactions,” Nucleic Acids Res. 28(2):e5 (2000)). In such methods, bead-bound primers are used to amplify and covalently immobilize one or more DNA amplicons simultaneously. This method results in the ability to more easily manipulate sequences and eliminates the need to conduct extensive PCR product purification steps. It further allows for the use of the PCR products in subsequent applications; i.e., beyond the confines of a test tube, glass slide, or microtiter plate.

Solid-phase PCR (“SP-PCR”) is a variation of the standard PCR method. SP-PCR can be used in a variety of applications and can overcome some of the problems associated with standard PCR protocols. There are several types of SP-PCR protocols. Some SP-PCR methods involve attaching the PCR products to a solid substrate after PCR amplification. Other protocols involve attaching a primer to the solid substrate and then conducting PCR amplification, resulting in bound amplicons. As described in Andreadis et al., “Use of Immobilized PCR Primers to Generate Covalently Immobilized DNAs for In Vitro Transcription/Translation Reactions”, Nucleic Acids Res. 28(2):e5 (2000), assays that involve post-PCR immobilization of the amplicons are available for numerous applications, including, without limitation, the following: the detection of single or multiple nucleotide polymorphisms (Lockley et al., “Colorimetric Detection of Immobilised PCR Products Generated on a Solid Support,” Nucleic Acids Res. 25(6):1313-1314 (1997); Saiki et al., “Genetic Analysis of Amplified DNA with Immobilized Sequence-Specific Oligonucleotide Probes,” Proc. Natl Acad. Sci. USA 86(16):6230-6234 (1989)); the identification of bacterial agents (Rasmussen et al., “Combined Polymerase Chain Reaction-Hybridization Microplate Assay Used to Detect Bovine Leukemia Virus and Salmonella,” Clin. Chem. 40:200-205 (1994); Oroskar et al., “Detection of Immobilized Amplicons by ELISA-Like Techniques,” Clin. Chem. 42:1547-1555 (1996)); genetic phylogeny analysis and hybridization assays for gene detection (Drobyshev et al., “Sequence Analysis by Hybridization with Oligonucleotide Microchip: Identification of β-Thalassemia Mutations,” Gene 188(1):45-52 (1997); Keller et al., “Detection of Human Immunodeficiency Virus Type 1 DNA by Polymerase Chain Reaction Amplification and Capture Hybridization in Microtiter Wells,” J. Clin. Microbiol. 29(3):638-641 (1991); Chevrier et al., “Rapid Detection of Salmonella Subspecies I by PCR Combined with Non-Radioactive Hybridisation Using Covalently Immobilised Oligonucleotide on a Microplate,” FEMS Immunol. Med. Microbiol. 10(3-4):245-501 (1995); Kohsaka, et al., “Solid-Phase Polymerase Chain Reaction,” J. Clin. Lab. Anal. 8:452-455 (1994)); in vitro transcription (Marble et al., “RNA Transcription from Immobilized DNA Templates,” Biotechnol. Prog. 11(4):393-396 (1995); Liu et al., “In Vitro Transcription on DNA Templates Immobilized to Streptavidin MagneSphere® Paramagnetic Particles,” Promega Notes Mag. 64:21-25 (1997)); and the development of cDNA microarrays for analysis of gene expression (Schena et al., “Quantitative Monitoring of Gene Expression Patterns with a Complementary DNA Microarray,” Science 270(5235):467-470 (1995); Schena et al., “Parallel Human Genome Analysis: Microarray-Based Expression Monitoring of 1000 Genes,” Proc. Natl Acad. Sci. USA 93(20):10614-10619 (1996)). Using these protocols, very small amounts of target nucleic acid molecules can be amplified and, therefore, detected using detection labels. For example, immobilization of the amplicons onto solid substrates can be combined with colorimetric or fluorescent signal generating labels, thereby facilitating the identification and quantification of the target nucleic acid molecules in a sample.

A major difference between standard PCR and SP-PCR procedures, is that in standard PCR protocols the oligonucleotide primers bind to template or target nucleic acid molecules in solution, while in SP-PCR protocols template or target nucleic acid molecules are hybridized to immobilized primers.

Microplate-based solid-phase extension products are usually detected by enzymatic assays (Koch et al., “Photochemical Immobilization of Anthraquinone Conjugated Oligonucleotides and PCR Amplicons On Solid Surfaces,” Bioconjugate Chem. 11:474-483 (2000); Kohsaka, et al., “Solid-Phase Polymerase Chain Reaction,” J. Clin. Lab. Anal. 8:452-455 (1994); Rasmussen, et al., “Combined Polymerase Chain Reaction-Hybridization Microplate Assay Used to Detect Bovine Leukemia Virus and Salmonella,” Clin. Chem. 40:200-205 (1994); Oroskar, et al., “Detection of Immobilized Amplicons by ELISA-Like Techniques,” Clin. Chem. 42:1547-1555 (1996)). For some applications, however, it would be preferable to employ direct detection of fluorescent products, which would allow quantitative estimation of yield over a wide dynamic range, as well as having the advantages of simplicity, flexibility, and cost. Thus, it would facilitate the use of microplate-based SP-PCR in high-throughput, automated applications. However, SP-PCR yields have not been sufficient for direct fluorescence detection with standard plate readers, requiring ˜100 femtomoles (fmol) of product per microplate well for reliable quantification.

Despite SP-PCR's potential, current SP-PCR protocols have serious practical limitations. Previous studies have demonstrated that steric hindrance inhibits the hybridization of DNA in solution to immobilized oligonucleotides (Shchepinov, et al., “Steric Factors Influencing Hybridization of Nucleic Acid Molecules to Oligonucleotide Arrays,” Nucleic Acids Res. 25:1155-1161 (1997); Guo, et al., “Direct Fluorescence Analysis of Genetic Polymorphisms by Hybridization With Oligonucleotide Arrays on Glass Supports,” Nucleic Acids Res. 22:5456-5465 (1994)). Steric hindrance can also affect solid-phase polymerization by impeding the attachment of Taq polymerase to tethered oligonucleotides that directly abut the supporting surface. It has been demonstrated that SP-PCR efficiency is enhanced when a polydeoxythymidine (“(dT)”) spacer is included at the 5′ end of the solid-phase primer (Oroskar, et al., “Detection of Immobilized Amplicons by ELISA-Like Techniques,” Clin. Chem. 42:1547-1555 (1996); Adessi, et al., “Solid Phase DNA Amplification: Characterization of Primer Attachment and Amplification Mechanisms,” Nucleic Acids Res. 28:87e (2000); Sjöroos, et al., “Solid-Phase PCR With Hybridization and Time-Resolved Fluorometry -for Detection of HLA-B27,” Clin. Chem. 47:498-504 (2001)). Solid-phase oligonucleotides containing 5′ (dT)ₙ spacers are desirable, because they are inexpensive and easy to synthesize. However, high background signals are often observed when using these primers to amplify AT-rich plant DNA templates.

The present invention is directed to overcoming the deficiencies in the prior art.

### SUMMARY OF THE INVENTION

The present invention is directed to a method for detecting a target nucleic acid molecule in a sample. A first oligonucleotide primer coupled by a linking agent to a solid substrate is provided, where the first oligonucleotide primer is complementary to at least 18 contiguous nucleic acid residues of a first strand of a target nucleic acid molecule. The first oligonucleotide primer is contacted with the sample under conditions effective to permit any of the first strand of the target nucleic acid molecule present in the sample to hybridize to the first oligonucleotide primer. The first oligonucleotide primer, after being hybridized to the first strand of the target nucleic acid molecule, is extended under conditions effective to yield a double stranded extension product coupled by the linking agent to the solid substrate; the linking agent is configured to position the first oligonucleotide primer sufficiently apart from the solid substrate to permit the extension. The extension product is denatured under conditions effective to yield an immobilized extension portion complementary to the target nucleic acid molecule. The immobilized extension portion is contacted with a detection probe, having a nucleotide sequence like that of the target nucleic acid molecule and a label, under conditions effective to permit the detection probe to hybridize specifically to the immobilized extension portion. Detection of the label immobilized on the solid substrate indicates the presence of the target nucleic acid molecule in the sample.

In one aspect of the invention, the entire assay may take place on a single reaction substrate, without the need to transfer the nucleic acid molecules, thereby greatly decreasing the occurrence of contamination. Further, the assay is suitable for automation, in that the hybridization, washing, and detection steps can be performed on the same solid substrate (e.g., in a single microtiter well). The assay may also be combined with PCR techniques to yield solid-phase amplification products. Thus, direct detection of the solid-phase amplification products should now provide a simple, reliable, quantitative, and cost effective means of sample analysis in a variety of molecular applications.

The present invention also results in increased extension of tethered oligonucleotides relative to reported values based on other current protocols. Additionally, the assay of the present invention may be used to achieve greater percentages of extension of the covalently bound primers, thereby resulting in a substantial improvement over estimates of other assays.

The present invention may be used in a variety of applications, including, without limitation, genotyping, disease diagnostics, pathogen detection, and gene expression studies.

### BRIEF DESCRIPTION OF THE DRAWINGS

FIGS. 1A-K are schematic drawings demonstrating, in sequence, one embodiment of the method for detecting a target nucleic acid molecule in accordance with the present invention.

FIG. 2 illustrates the Arabidopsis thaliana phytochrome C (PhyC) gene (3,572 transcribed bp), and the molecules used in this study derived from Exon I: the 251 bp PCR product, the synthetic 80-mer (80^{fl}), and the R^{tr} probe. The HpaII restriction site in 80 ^{fl} was introduced by modifying one nucleotide of the genomic sequence, while the HpaII site in the PCR product is native to the Columbia allele. Fluorescent labels are depicted as either dark (i.e., black) or light (i.e., grey) circles. The light-colored circle represents fluorescein (R^{fl}). The dark-colored circle represents Texas red (R^{tr}).

FIG. 3 illustrates a hybridization and extension assay for determining optimal spacer length for tethered oligonucleotides. The experiment consisted of four trials with eight 8-well strips per trial, and two wells per treatment per strip with random placement of treatments within strips. The amount of covalently bound primer was determined for one strip per trial using YOYO-1 iodide. 5′ amino-modified primers with HEG spacers of varying lengths (F^{(HEG)}ₙ) were tethered to microwells. The amount of primer tethered in one 8-well strip per trial was determined by YOYO-1 binding. For seven strips, wells were hybridized to a fluorescein-labeled synthetic 80-mer (80^{fl}). The light-colored (i.e., grey) circle represents the fluorescein label. Unhybridized 80^{fl} was removed by washing, the quantity of remaining 80^{fl} was measured, and the tethered primers were extended by Taq polymerase. Double-stranded solid-phase extension products were digested with HpaII, and the liquid-phase fragments were then transferred to a microplate and quantified. For selected wells, samples were concentrated and the presence of the expected 54 bp restriction fragment was confirmed on a DNA sequencer by electrophoresis.

FIGS. 4A-B show the result from experiments for verification and quantification of SP-PCR. These experiments consisted of three trials with four 8-well strips per trial and one treatment per strip. The concentrations of Taq polymerase and tethered oligonucleotides were varied in each treatment, and the amount of covalently bound primer was determined for one well per strip using YOYO-1. Fluorescein-labeled double-stranded products were generated by inclusion of liquid-phase R^{fl} primer. The light-colored (i.e., grey) circle represents the R^{fl} fluorescein label. In FIG. 4A, solid-phase extension was confirmed in selected wells by HpaII digestion of tethered double-stranded DNA and visualization of the resulting 161 bp fragment on a denaturing polyacrylamide gel. In FIG. 4B, solid-phase extension was quantified by denaturation of tethered double-stranded products, washing, and hybridization of a fluorescent probe (R^{tr}) complementary to the 3′ end of the single-stranded product. The dark-colored (i.e., black) circle represents the R^{tr} Texas red label. Wells were washed and fluorescence quantified by comparison to a standard. The amount of fluorescein-labeled complementary strand (and/or R^{fl} liquid-phase primer) was also determined after: completion of SP-PCR, denaturation, probing, and additional washing.

FIGS. 5A-D show hybridization and solid-phase extension of tethered oligonucleotides with 5′ HEG spacers of various lengths. Error bars represent 2× standard errors. FIG. 5A shows the quantity of fluorescent label (in fmol) after hybridization of 80^{fl} to tethered primers (solid line), and after primer extension (dashed line), the latter measured as the quantity of labeled liquid-phase restriction fragment. Results are shown for spacers with 0, 5, 10, and 20 HEG residues. FIG. 5B shows the percent efficiency of extension (extension×100/hybridization) shown in FIG. 5A. FIG. 5C shows the quantity of probe hybridized (solid line) and extended (dashed line) for spacers with 1-8 HEG residues, as in FIG. 5A. FIG. 5D shows the efficiency of extension for reactions shown in FIG. 5C.

FIGS. 6A-B provide electropherogram results showing the HpaII restriction fragment from SP-PCR and residual full-length product from a liquid-phase PCR “control”. The x-axis represents the size of the fragments, measured in base pairs (bps). The y-axis represents the fluorescence intensity of the fragments. Light (i.e., grey) lines denote internal size standards. Dark, solid lines denote fluorescein-labeled products. FIG. 6A shows experimental well containing tethered F^{(HEG)}₅ oligonucleotide, Taq, and all other PCR components. The arrow denotes the 161 bp HpaII restriction fragment from cleavage of double-stranded SP-PCR products. FIG. 6B shows a liquid-phase PCR control well containing all reactants except tethered oligo. The arrow indicates a weak signal at 251 bp representing residual, full-length, liquid-phase PCR product (not cleaved by HpaII).

### DETAILED DESCRIPTION OF THE INVENTION

The present invention is directed to a method for detecting a target nucleic acid molecule in a sample. A first oligonucleotide primer coupled by a linking agent to a solid substrate is provided, where the first oligonucleotide primer is complementary to at least 18 contiguous nucleic acid residues of a first strand of a target nucleic acid molecule. The first oligonucleotide primer is contacted with the sample under conditions effective to permit any of the first strand of the target nucleic acid molecule present in the sample to hybridize to the first oligonucleotide primer. The first oligonucleotide primer, after being hybridized to the first strand of the target nucleic acid molecule, is extended under conditions effective to yield a double stranded extension product coupled by the linking agent to the solid substrate; the linking agent is configured to position the first oligonucleotide primer sufficiently apart from the solid substrate to permit the extension. The extension product is denatured under conditions effective to yield an immobilized extension portion complementary to the target nucleic acid molecule. The immobilized extension portion is contacted with a detection probe, having a nucleotide sequence like that of the target nucleic acid molecule and a label, under conditions effective to permit the detection probe to hybridize specifically to the immobilized extension portion. Detection of the label immobilized on the solid substrate indicates the presence of the target nucleic acid molecule in the sample.

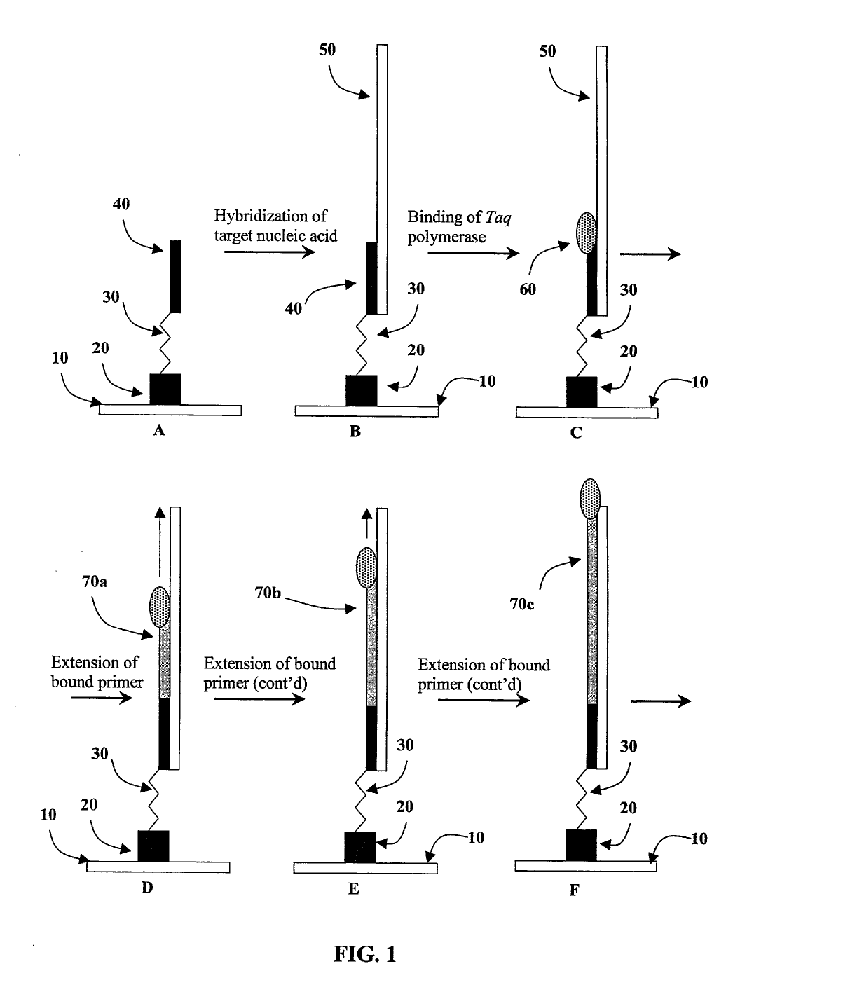

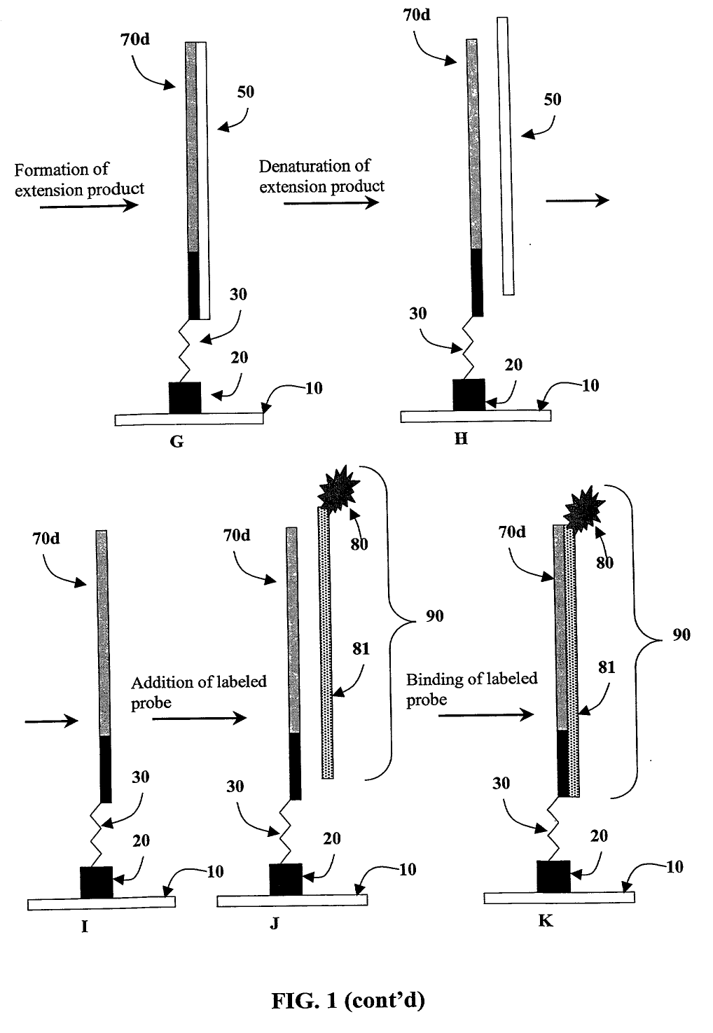

FIGS. 1A-K depict one embodiment of the assay of the present invention. As shown in FIG. 1A, first oligonucleotide primer 40, which is specific to one strand of a target nucleic acid molecule, is coupled by a linking agent to solid substrate 10. The linking agent comprises 6-amino-phosphohexane 20 linked through a phosphodiester bond to a hexaethyleneglycol spacer 30. These components of the linking agent are generated using a 5′-Amino Modifier C6 spacer and phosphoramidite spacer. The hexaethyleneglycol spacer 30 is coupled to the 5′-end of first oligonucleotide primer 40, and the 6-amino-phosphohexane 20 is covalently bound to solid substrate 10 (e.g., through standard carbodiimide condensation chemistry, as described infra).

A sample to be analyzed for the presence of the target nucleic acid molecule is added under conditions that would allow the 3′-end of one of the strands of target nucleic acid molecule 50 to hybridize to the bound first oligonucleotide primer 40, as shown in FIG. 1B. Once target nucleic acid molecule 50 is hybridized to first oligonucleotide primer 40, Taq polymerase 60 is added under conditions to allow it to bind to target nucleic acid molecule 50, as shown in FIG. 1C, and then to extend first oligonucleotide primer 40. FIGS. 1D-F show the extension of first oligonucleotide primer 40 from the 5′-end to the 3′-end to yield a complementary strand to target nucleic acid molecule 50. This is depicted, in sequence, as strands 70a-d in FIGS. 1D, 1E, 1F, and 1G, respectively. The resulting double-stranded extension product—comprising the strand of target nucleic acid molecule 50 hybridized to fully extended first oligonucleotide primer 70d—is then subjected to conditions effective to allow the extension product to become denatured (FIG. 1H), leaving fully extended first oligonucleotide primer 70d that is covalently immobilized on solid substrate 10 (FIG. 1I). Immobilized, extended first oligonucleotide primer 70d is then used as a template for a detection probe 90. Probe 90 comprises a nucleic acid molecule 81 that is complementary to the extended oligonucleotide primer and a label (e.g., a fluorescent moiety) 80 that is coupled to the 5′-end of the nucleic acid molecule 81 (FIG. 1J). As shown in FIG. 1K, immobilized, extended first oligonucleotide primer 70d and detection probe 90 are incubated under conditions effective to allow for their hybridization. Any unbound nucleic acid molecules and detection probe units are washed from solid substrate 10, as described infra, allowing detection probes 90 that remain hybridized to be immobilized and the extended first oligonucleotide primer 70d to be detected and quantified.

In one embodiment of the present invention, the assay may be carried out in the form of a solid-phase polymerase chain reaction (“SP-PCR”) procedure. This involves the same general reaction components as standard polymerase chain reaction (“PCR”) procedures previously described in the art (U.S. Pat. No. 4,683,195, U.S. Pat. No. 4,683,202, and U.S. Pat. No. 4,800,159, which are hereby incorporated by reference in their entirety), except that one of the oligonucleotide primers of each pair of primers is modified to allow for attachment to a solid substrate (e.g., a well of a microtiter plate). General SP-PCR protocols known in the art may be used in the present invention. Examples of references that generally describe SP-PCR protocols include Kohsaka et al., “Solid-Phase Polymerase Chain Reaction,” J. Clinical Lab. Anal. 8:452-455 (1994) and Adessi et al., “Solid Phase DNA Amplification: Characterisation of Primer Attachment and Amplification Mechanisms,” Nucleic Acids Res. 28(20):e87 (2000), which are hereby incorporated by reference in their entirety.

In one aspect of the present invention, the PCR reaction mixture includes the nucleic acid molecules sample, a pair of oligonucleotide primers (one primer being modified for covalent binding to the solid substrate), a mixture of deoxynucleotides (i.e., dATP, dCTP, dTTP, dGTP, dITP, dUTP), a heat-stable polymerase, and a buffer solution. Heat-stable polymerases that may be used with the present invention include, but are not limited to, the following polymerases: Thermus aquaticus DNA polymerase (Taq polymerase); Thermus thermophilus DNA polymerase; E. coli DNA polymerase; T4 DNA polymerase; and Pyrococcus DNA polymerase.

The term “nucleic acids” as used herein is to be interpreted broadly and comprises deoxyribonucleic acids (DNA) and ribonucleic acids (RNA), including modified DNA and RNA, as well as other hybridizing nucleic acid-like molecules, such as peptide nucleic acid (PNA).

One aspect of the present invention involves the use of a solid substrate that is suitable for the covalent binding of a modified oligonucleotide primer to the surface of the solid substrate. The solid phase detection assay of the present invention can take place entirely in a single reaction compartment or solid substrate. A variety of types of compartments/solid substrates may be used, including, without limitation, the following: cellulose; nitrocellulose; nylon membranes; controlled-pore glass beads; acrylamide gels; polystyrene matrices; activated dextran; avidin/streptavidin-coated polystyrene beads; agarose; polyethylene; functionalized plastic, glass, silicon, aluminum, steel, iron, copper, nickel, and gold; tubes; wells; microtiter plates or wells; slides; discs; columns; beads; membranes; well strips; films; chips; and composites thereof. In one embodiment, prior to its use in the detection assay of the present invention, a portion of the surface of the solid substrate is coated with a chemically functional group to allow for covalent binding of the solid phase primer to the surface of the solid substrate. Solid substrates with the functional group already included on the surface are commercially available. In addition, the functional groups may be added to the solid substrates by the practitioner.

A number of methods may be used to couple the oligonucleotide primer to the solid substrate, including, without limitation, the following methods: covalent chemical attachment; biotin-avidin/streptavidin; and UV irradiation (Conner et al., “Detection of Sickle Cell β S-Globin Allele by Hybridization with Synthetic Oligonucleotides,” Proc. Natl Acad. Sci. USA 80(1):278-282 (1983); Lockley et al., “Colorimetric Detection of Immobilised PCR Products Generated on a Solid Support,” Nucleic Acids Res. 25(6):1313-1314 (1997), which are hereby incorporated by reference in their entirety).

The primer/solid substrate linkages may include, without limitation, the following linkage types: disulfide; carbamate; hydrazone; ester; (N)-functionalized thiourea; functionalized maleimide; streptavidin or avidin/biotin; mercuric-sulfide; gold-sulfide; amide; thiolester; azo; ether; and amino.

The solid substrate may be functionalized with a number of different functional groups, including without limitation, the following: olefin; amino; hydroxyl; silanol; aldehyde; keto; halo; acyl halide; or carboxyl. The solid substrate may be functionalized with an amino group by reacting it with any of the following amine compounds: 3-aminopropyl triethoxysilane; 3-aminopropylmethyldiethoxysilane; 3-aminopropyl dimethylethoxysilane; 3-aminopropyl trimethoxysilane; N-(2-aminoethyl)-3-aminopropylmethyl dimethoxysilane; N-(2-aminoethyl-3-aminopropyl) trimethoxysilane; aminophenyl trimethoxysilane; 4-aminobutyldimethyl methoxysilane; 4-aminobutyl triethoxysilane; aminoethylaminomethylphenethyl trimethoxysilane; and mixtures thereof. Further, an olefin-containing silane may be used and may include: 3-(trimethoxysilyl)propyl methacrylate; N-[3-(trimethoxysilyl)propyl]-N′-(4-vinylbenzyl)ethylenediamine; triethoxyvinylsilane; triethylvinylsilane; vinyltrichlorosilane; vinyltrimethoxysilane; vinyltrimethylsilane; and mixtures thereof. Another aspect of the invention includes a solid substrate that is functionalized with a silanol polymerized with an olefin-containing monomer. The olefin-containing monomer may contain any of the following functional groups: acrylic acid; methacrylic acid; vinylacetic acid; 4-vinylbenzoic acid; itaconic acid; allyl amine; allylethylamine; 4-aminostyrene; 2-aminoethyl methacrylate; acryloyl chloride; methacryloyl chloride; chlorostyrene; dichlorostyrene; 4-hydroxystyrene; hydroxymethylstyrene; vinylbenzyl alcohol; allyl alcohol; 2-hydroxyethyl methacrylate; poly(ethylene glycol) methacrylate; and mixtures thereof.

If the solid substrate is made of a polymer, it can be produced from any of the following monomers: acrylic acid; methacrylic acid; vinylacetic acid; 4-vinylbenzoic acid; itaconic acid; allyl amine; allylethylamine; 4-aminostyrene; 2-aminoethyl methacrylate; acryloyl chloride; methacryloyl chloride; chlorostyrene; dischlorostyrene; 4-hydroxystyrene; hydroxymethyl styrene; vinylbenzyl alcohol; allyl alcohol; 2-hydroxyethyl methacrylate; poly(ethylene glycol) methacrylate; and mixtures thereof, together with one of the following monomers: acrylic acid; acrylamide; methacrylic acid; vinylacetic acid; 4-vinylbenzoic acid, itaconic acid; allyl amine; allylethylamine; 4-aminostyrene; 2-aminoethyl methacrylate; acryloyl chloride; methacryloyl chloride; chlorostyrene; dichlorostyrene; 4-hydroxystyrene; hydroxymethyl styrene; vinylbenzyl alcohol; allyl alcohol; 2-hydroxyethyl methacrylate; poly(ethylene glycol) methacrylate; methyl acrylate; methyl methacrylate; ethyl acrylate; ethyl methacrylate; styrene; 1-vinylimidazole; 2-vinylpyridine; 4-vinylpyridine; divinylbenzene; ethylene glycol dimethacrylate; N,N′-methylenediacrylamide; N,N′-phenylenediacrylamide; 3,5-bis(acryloylamido) benzoic acid; pentaerythritol triacrylate; trimethylolpropane trimethacrylate; pentaerytrithol tetraacrylate; trimethylolpropane ethoxylate (14/3 EO/OH) triacrylate; trimethylolpropane ethoxylate (7/3 EO/OH) triacrylate; trimethylolpropane propoxylate (1 PO/OH) triacrylate; trimethylolpropane propoxylate (2 PO/OH) triacrylate; and mixtures thereof.

In carrying out the present invention, oligonucleotide primers that are specific to the target nucleic acid molecule are used. These primers can be in the form of DNA, RNA, PNA (i.e., peptide nucleotide analogs), and DNA/RNA composites. Typically, these primers have lengths ranging from 8 to 30 nucleotides. A pair of primers is made for each target nucleic acid molecule of interest.

As described in Adessi et al., “Solid Phase DNA Amplification: Characterization of Primer Attachment and Amplification Mechanisms,” Nucleic Acids Res. 28:e87 (2000), which is hereby incorporated by reference in its entirety, once attached to the solid substrate, it is preferable that the solid phase primer/solid substrate interface have two characteristics: (1) the surface density should be high enough for detecting immobilized nucleic acid molecule amplification products by hybridization assay after SP-PCR; and (2) the coupling (e.g., covalent linkage) between the solid phase primer and the solid substrate should not be affected by the repeated heating and cooling cycles during the nucleic acid molecule amplification procedure.

Preferably, the oligonucleotide primers are modified and linked to the solid substrate in such a way as not to hinder the ability of the polymerase to access to the primer and to extend the primer from its 3′ end. A number of different linking methods may be employed (Adessi et al., “Solid Phase DNA Amplification: Characterisation of Primer Attachment and Amplification Mechanisms,” Nucleic Acids Res. 28(20):e87 (2000) and U.S. Pat. No. 5,700,642, which are hereby incorporated by reference in their entirety). Generally, the linking occurs between a reactive site on the solid substrate and a reactive site on the 5′-end of the modified oligonucleotide. Alternatively, the linking may occur between a reactive site on the solid substrate and a reactive site on the 3-end of the modified oligonucleotide. In one embodiment the reaction is between a 5′-thiol-modified oligonucleotide attached to amino-silanised glass slides using a heterobifunctional cross-linker reagent (Adessi et al., “Solid Phase DNA Amplification: Characterisation of Primer Attachment and Amplification Mechanisms,” Nucleic Acids Res. 28(20):e87 (2000), which is hereby incorporated by reference in its entirety).

In one embodiment, in order to link the oligonucleotide primer to the solid substrate, the oligonucleotide can be modified at its 5′-end. Modification of oligonucleotides can be achieved through phosphorylation (e.g., phosphoramidites), amination, thiolation, conjugation, or spacer (e.g., polyethylene glycol or phosphoramidites) introduction. A number of 5′-terminus modifiers are commercially available from Glen Research Corporation (Sterling, Va.), including: 5′-Amino-Modifier C3-TFA; 5′-Amino-Modifier C6; 5′-Amino-Modifier C6-TFA; PC Amino-Modifier Phosphoramidite; 5′-Amino-Modifier C12; 5′-Thiol-Modifier C6; 5′-Amino-Modifier 5; and Thiol-Modifier C6 S-S. The terminus modifiers can be combined with spacer modifiers. Examples of commercially available phosphoramidite spacers from Glen Research Corporation (Sterling, Va.), include the following: Spacer Phosphoramidite 9; Spacer Phosphoramidite C3; dSpacer CE Phosphoramidite; Spacer Phosphoramidite 18; Spacer C12 CE Phosphoramidite; PC Spacer Phosphoramidite; 3′-Spacer C3 CPG; 3′-Spacer 9 CPG; and Abasic Phosphoramidite. Other linkers may be used, including poly(dT) linkers.

In another embodiment of the present invention, the 5′-end is modified using amino modifiers. Examples include the following: the modified nucleoside phosphoramidite Amino-Modifier-dT (Glen Research, Sterling Va.), which contains a base labile trifluoroacetyl group that protects the primary amine attached to thymine via a 10-atom spacer arm; phosphoramidite 5′-Amino-Modifier C6 (Glen Research, Sterling Va.), which contains a primary amino group protected with an acid labile monomethoxytrityl group; and N-trifluoroacetyl-6-aminohexyl-2-cyanoethyl N′,N′-isopropylphosphoramidite (Applied Biosystems, Foster City, Calif.). The amino-containing oligonucleotide primers are usually prepared by standard phosphoramidite chemistry, although any other method resulting in the oligonucleotides containing primary amine groups may also be used. In a preferred embodiment of the present invention, the 5′-amino modifier is a 5′-Amino Modifier C6 spacer (Glen Research Corporation, Sterling, Va.) comprising the following chemical structure:

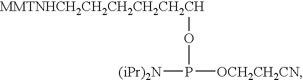

and having the chemical formula C₃₅H₄₈N₃O₃P and the proper name: 6-(4-monomethoxytritylamino)hexyl-(2-cyanoethyl)-(N,N-diisopropyl)-phosphoramidite.

Amino-modified oligonucleotides are especially useful, because they may be easily transformed to the corresponding carboxyl- or thiol-terminated derivatives for use in immobilization or spacer arm attachment reactions requiring 5′-functionalities other than amino. These modified oligonucleotides may also be converted to the corresponding carboxyl derivatives by reaction with succinic anhydride (Bischoff et al., “Introduction of 5′-Terminal Functional Groups Into Synthetic Oligonucleotides for Selective Immobilization,” Anal. Biochem. 164:336-344 (1987), which is hereby incorporated by reference in its entirety). Further, the carboxyl-derivatized oligonucleotide primer may be linked to a bifunctional linker (e.g., 1,6-diaminohexane) prior to attachment to the solid substrate, which can be completed by a coupling reaction in the presence of an activating agent such as a water soluble carbodiimide. As described in U.S. Pat. No. 5,700,642 to Monteforte et al., which is hereby incorporated by reference in its entirety, when using thiol-modified oligonucleotides, these modifications may be made by treating the unprotected 5′-amino group of a functionalized oligonucleotide primer with dithiobis(succinimidylpriopionate), followed by sulfhydryl deprotection with dithioerythritol (Bischoff et al., “Introduction of 5′-Terminal Functional Groups Into Synthetic Oligonucleotides for Selective Immobilization,” Anal. Biochem. 164:336-344 (1987), which is hereby incorporated by reference in its entirety).

In another aspect of the present invention, the linking agent includes a spacer between the amino modifier and the oligonucleotide solid phase primer. Spacer molecules are used to maximize the sensitivity and efficiency of the detection assay. One of the major roles that spacers play in the process of the present invention is to reduce the steric hindrance on the linking of the solid phase oligonucleotide primer to the solid substrate and the hybridization of the target nucleic acid molecule to the immobilized primer and the subsequent nucleic acid molecule synthesis using polymerase. Spacers have been shown to be effective in enhancing the hybridization behavior of immobilized oligonucleotides (Shchepinov et al., “Steric Factors Influencing Hybridisation of Nucleic Acids to Oligonucleotide Arrays,” Nucleic Acids Res. 25(6):1155-1161 (1997), which is hereby incorporated by reference in its entirety).

The spacer may comprise either a nucleoside or non-nucleoside spacer. Generally, the spacer is a monomeric molecule that can be added as units. Spacers may be prepared using a variety of monomeric units and by condensation of these units onto an amine-functionalized solid substrate (e.g., polypropylene). One method of adding spacer units to the oligonucleotide primer is by using standard phosphoramidite chemistry.

In another embodiment of the present invention, the spacer is a polyethylene glycol spacer. The polyethylene glycol spacer may comprise, without limitation, units of either triethylene glycol, hexaethylene glycol, or heptaethylene glycol. In one specific embodiment, the spacer comprises up to 0 to 20 molecules of hexaethylene glycol linked by a phosphodiester bond generated through the use of Spacer Phosphoramidite 18 (Glen Research Corporation, Sterling, Va.), having the following structure:

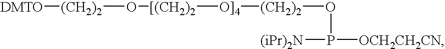

and having the chemical formula C₄₂H₆₁N₂O₁₀P and the proper name: 18-O-Dimethoxytrityl-hexaethyleneglycol, 1-[(2-cyanoethyl)-(N,N-diisopropyl)]-phosphoramidite. Hexaethylene glycol is referred to herein as “HEG.” The expression “(HEG)ₙ” is used to describe the number of HEG molecules used in the spacer; the “n” represents the number of HEG molecules. For example, (HEG)₅ signifies an HEG spacer comprising 5 HEG molecules.

In the present invention, the linking agent is configured to position the oligonucleotide primers sufficiently apart from the solid substrate to permit binding of the polymerase (e.g., Taq polymerase) and extension of the primer. Preferably, the length of the linking agent may range from about 5 to about 500 Ångstroms, preferably from about 25 to about 250 Ångstroms.

The presence of the target nucleic acid molecule in the test sample can be detected using a variety of detection labels. In one embodiment, the detection probe has a hybridization temperature of 20-85° C. The labels may include without limitation, the following labeling agents: chromophores; fluorescent dyes; enzymes; antigens; heavy metals; magnetic probes; dyes; phosphorescent groups; radioactive materials; chemiluminescent moieties; and electrochemical detecting moieties.

In one embodiment, the detection probes are designed to hybridize to the immobilized extension product under stringent conditions. Less stringent conditions may also be selected. Generally, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. The Tₘ is dependent upon the solution conditions and the base composition of the probe, and for DNA:RNA hybridization may be estimated using the following equation:

Tₘ=79.8° C.+(18.5×Log [Na+])+(58.4° C.×%[G+C])−(820/#bp in duplex)−(0.5×% formamide)

Promega Protocols and Applications Guide, 2d ed., Promega Corp., Madison, Wis. (1991), which is hereby incorporated by reference in its entirety.

Generally, suitable stringent conditions for nucleic acid hybridization assays or gene amplification detection procedures are as set forth above or as identified in Southern, “Detection of Specific Sequences Among DNA Fragments Separated by Gel Electrophoresis,” J. Mol. Biol., 98:503-17 (1975), which is hereby incorporated by reference in its entirety. For example, conditions of hybridization at 42° C. with 5× SSPE (saline sodium phosphate EDTA buffer) and 50% formamide with washing at 50° C. with 0.5× SSPE can be used with a nucleic acid probe containing at least 20 bases, preferably at least 25 bases or more preferably at least 30 bases. Stringency may be increased, for example, by washing at 55° C. or more preferably 60° C. using an appropriately selected wash medium having an increase in sodium concentration (e.g., 1× SSPE, 2× SSPE, 5× SSPE, etc.). If problems remain with cross-hybridization, further increases in temperature can also be selected, for example, by washing at 65° C., 70° C., 75° C., or 80° C. By adjusting hybridization conditions, it is possible to identify sequences having the desired degree of homology (i.e., greater than 80%, 85%, 90%, or 95%).

Various types of labels can be used in the present invention, including, without limitation, labels comprising chromophores, fluorescent dyes, enzymes, antigens, heavy metals, magnetic probes, dyes, phosphorescent groups, radioactive materials, chemiluminescent moieties, or electrochemical detecting moieties.

A number of amine-reactive fluorescent dyes are available for use in the present invention. These dyes can be purchased from a number of different commercial vendors, and used singly or in combination. Examples of fluorophores that are available include, but are not limited to, the following: AMCA-S; AMCA; BODIPY 493/503; BODIPY FL; BODIPY FL Br₂; BODIPY R6G; BODIPY 530/550; BODIPY TMR; BODIPY 558/568; BODIPY 564/570; BODIPY 576/589; BODIPY 581/591; BODIPY TR; Cascade Blue; CI-NERF; Dansyl; Dialkylamino-coumarin; 4′,5′-Dichloro-2′,7′-dimethoxy-fluorescein; 2′,7′-Dichloro-fluorescein; DM-NERF; Eosin; Eosin F₃S; Erythrosin; Fluorescein; Hydroxycoumarin; Isosulfan blue; Lissamine rhodamine B; Malachite green; Methoxycoumarin; Naphthofluorescein; NBD; Oregon Green 488; Oregon Green 500; Oregon Green 514; PyMPO; Pyrene; Rhodamine 6G; Rhodamine Green; Rhodamine Red; Rhodol Green; 2′,4′,5′,7′-Tetrabromo-sulfonefluorescein; Tetramethyl-rhodamine (TMR); Texas Red; and X-rhodamine (all of the foregoing are commercially available from Molecular Probes, Inc., Eugene, Oreg.). Other commercially available dyes may be used, including allophycocyanin, propidium iodide, Cy5, and derivatives of these dyes.

The method of the present invention may be used broadly in a variety of applications, including, without limitation, pathogen detection, disease diagnostics, genotyping, and expression studies. For example, the present invention may be used to detect target nucleic acid molecules such as gene loci from any organism having either DNA or RNA as its genetic information. The organism of interest may include, without limitation, humans, animals, plants, fungi, bacteria, and viruses.

In one embodiment, the method of the present invention is used to detect infectious diseases caused by bacterial, viral, parasitic, and fungal infectious agents. Bacterial diseases that may be detected using the present invention include, without limitation, diseases caused by the following bacteria: Escherichia coli, Salmonella, Shigella, Klebsiella, Pseudomonas, Listeria monocytogenes, Mycobacterium tuberculosis, Mycobacterium avium-intracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasma, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, and Actinomycetes.

Another embodiment involves the use of the assay of the present invention to detect infectious disease caused by a fungal infectious agent, including, without limitation, diseases caused by the following fungal agents: Cryptococcus neoformans, Blastomyces dermatitidis, Histoplasma capsulatum, Coccidioides immitis, Paracoccicioides brasiliensis, Candida albicans, Aspergillus fumigautus, Phycomycetes (Rhizopus), Sporothrix schenckii, Chromomycosis, and Maduromycosis.

A further embodiment involves the use of the assay of the present invention to detect infectious diseases caused by a viral infectious agent, including, without limitation, diseases caused by the following viral agents: human immunodeficiency virus, human T-cell lymphocytotrophic viurs, hepatitis viruses, Epstein-Barr Virus, cytomegalovirus, human papillomaviruses, orthomyxo viruses, paramyxo viruses, adenoviruses, corona viruses, rhabdo viruses, polio viruses, toga viruses, bunya viruses, arena viruses, rubella viruses, and reo viruses.

Yet a further embodiment involves the use of the assay of the present invention to detect infectious diseases caused by parasitic infectious agents, including, without limitation, those diseases caused by the following parasitic agents: Plasmodium falciparum, Plasmodium malaria, Plasmodium vivax, Plasmodium ovale, Onchoverva volvulus, Leishmania, Trypanosoma spp., Schistosoma spp., Entamoeba histolytica, Cryptosporidum, Giardia spp., Trichimonas spp., Balatidium coli, Wuchereria bancrofti, Toxoplasma spp., Enterobius vermicularis, Ascaris lumbricoides, Trichuris trichiura, Dracunculus medinesis, trematodes, Diphyllobothrium latum, Taenia spp., Pneumocystis carinii, and Necator americanis.

One aspect of the present invention involves the described assay to detect genetic diseases, including, without limitation, diseases such as: 21 hydroxylase deficiency, cystic fibrosis, Fragile X Syndrome, Turner Syndrome, Duchenne Muscular Dystrophy, Down Syndrome, heart disease, single gene diseases, HLA typing, phenylketonuria, sickle cell anemia, Tay-Sachs Syndrome, thalassemia, Klinefelter's Syndrome, Huntington's Disease, autoimmune diseases, lipidosis, obesity defects, hemophilia, inborn errors in metabolism, and diabetes.

Another embodiment involves the use of the described assay to detect cancer having a known nucleotide sequence and involving oncogenes, tumor suppressor genes, or genes involved in DNA amplification, replication, recombination, or repair. A further embodiment of the present invention includes the detection of cancers that are associated with a gene, including, without limitation, such genes and/or cancers as follows: BRCA1 gene, p53 gene, Familial polyposis coli, Her2/Neu amplification, Bcr/Ab1, K-ras gene, human papillomavirus Types 16 and 18, leukemia, colon cancer, breast cancer, lung cancer, prostate cancer, brain tumors, central nervous system tumors, bladder tumors, melanomas, liver cancer, osteosarcoma and other bone cancers, testicular and ovarian carcinomas, ENT tumors, and loss of heterozygosity.

One embodiment involves the use of the described assay to detect genetic polymorphisms used as markers for non-cancer genetic diseases of humans and animals (e.g., hip dysplasia in dogs), as well as for horticulturally and/or agronomically important traits in plant crops or in livestock (e.g., vitamin content).

A further aspect of the present invention involves the use of the solid phase assay in the area of environmental monitoring, forensics, and food and feed industry monitoring.

Another aspect of the present invention involves using the assay to detect a target nucleic acid molecule that is a gene locus of an organism having DNA as its genetic information. The gene locus may originate from a variety of organisms, including, without limitation, humans, animals, plants, fungi, bacteria, and viruses.

The present invention can also be used for genetic mapping. Genetic mapping is one of the core technologies of the genomics revolution in biology.

In one embodiment of the genetic mapping assay of the present invention, a pair of primers are synthesized for each locus to be mapped. One of each pair is modified at the 5′ end and tethered to the wells of a special microplate. DNA samples from the individuals to be genotyped are added to the wells and used as templates in solid-phase amplifications of the target locus. The target locus is separately amplified from the two parental sources in reactions that incorporate different fluorescent dyes. The parental amplicons are hybridized against the tethered amplicon under competitive conditions. Non-complementary dye-labeled parental amplicons are washed away and the fluorescent signal of the complementary allele is detected. This embodiment has a number of advantages: (1) genotyping is rapid and automatable; (2) assay costs, on a per locus basis, are significantly less than for other SNP detection strategies; and (3) the ability to quantify allelic dosage makes the assay suitable for a wide variety of different mapping populations.

Another embodiment of the present invention involves a genotyping assay comprising four basic steps: (1) amplification and fluorescent labeling of two or more probes; (2) solid-phase amplification of the unknown sequence using a tethered primer; (3) competitive hybridization of the probes to the unknowns under conditions of high specificity; and (4) detection of fluorescent label in the microtiter plate. The assay may be used for genotyping of any DNA sequences of several hundred base pairs for which PCR primers can be developed and which differ by a very small number of nucleotides. All amplification steps use a common pair of primers. This embodiment of the present invention is appropriate for a wide range of genotyping applications, especially where the nature of the sequence variation within the amplified molecules is unknown. The format can accommodate biallelic or multiallelic loci, compositional or length variation, and can be modified to screen multiple loci in individual wells. It can also be used to reduce the allelism of experimental populations or pedigrees with complex allelic heterogeneity. Further, the competitive hybridization assay portion of the invention can be used to measure the relative proportions of different alleles in a sample, thereby opening up a range of other applications, such as: (1) measuring the dosage of different allelic variants in a sample, as in tumour diagnosis; (2) measuring the number of copies of transgenes present in genetically modified tissue; and (3) determining the parental origin of genomes that derive from multiple sources (polyploidy) or that have undergone genome rearrangement events (such as aneuploidy).

### EXAMPLES

The Examples set forth below are for illustrative purposes only and are not intended to limit, in any way, the scope of the present invention.

### Example 1—Oligonucleotides

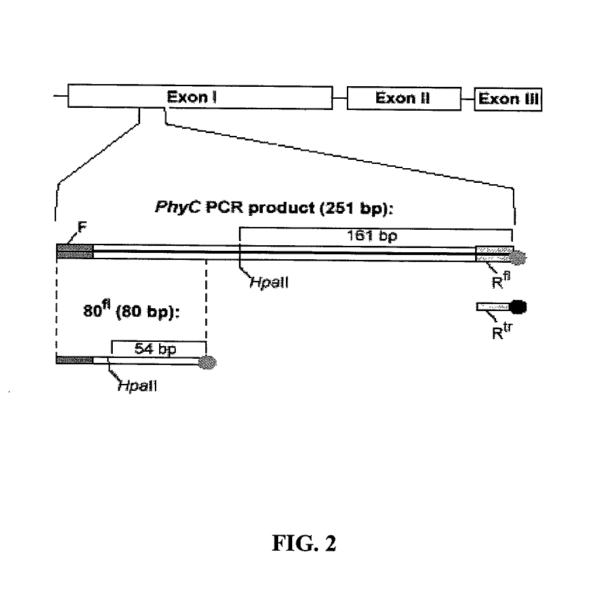

Primers and probes (Table 1) were derived from the first exon of the Arabidopsis thaliana phytochrome C (PhyC) gene (Cowl et al., “The PhyC Gene of Arabidopsis: Absence of the Third Intron Found in PhyA and PhyB,” Plant Physiol. 106:813-814 (1994), which is hereby incorporated by reference in its entirety). FIG. 2 shows the relative positions of oligonucleotides within PhyC.

TABLE 1
Oligonucleotides used for evaluation of solid-phase extension and
PCR.

| Oligo | 5′ modification | DNA sequence (5′-3′)¹ | Experimental Role |
|---|---|---|---|
| F^{(HEG)}ₙ | aminoC6²-HEG³ | GCC TTT TTA TGC GAT TCT GC | Tethered in |
| | | (SEQ.ID.No.1) | microwells. |
| F^{(dT)}₁₀ | aminoC6 | TTT TTT TTT TGC CTT TTT ATG CGA | Tethered in |
| | | TTC TGC | microwells. |
| | | (SEQ.ID.No.2) | |
| 80^{fl} | fluorescein | CAG GCA CCT CAT CAG GAC TCA CAG | Hybridized to |
| | | GAT CCA AAT CTA TAA CAA GAC CTT | tethered primers |
| | | CCT CAA TCC GGT GCA GAA TCG | (solid-phase |
| | | CAT AAA AAG GC | extension assay). |
| | | (SEQ.ID.No.3) | |
| F | none | GCC TTT TTA TGC GAT TCT GC | Liquid-phase primer |
| | | (SEQ.ID.No.4) | (SP-PCR). |
| R^{fl} | fluorescein | CGG GTA GGA GTA CCT TGA AT | Liquid-phase primer |
| | | (SEQ.ID.No.5) | (SP-PCR). |
| R^{tr} | Texas red⁴ | CGG GTA GGA GTA CCT TGA AT | Probe (SP-PCR). |
| | | (SEQ.ID.No.6) | |

¹80^{fl} contains a HpaII site (underlined) not found in the Arabidopsis genomic sequences.
²Amino modifier C6 (Glen Research, Sterling, Va., USA).
³0-20 molecules of hexaethyleneglycol (Spacer Phosphoramidite 18, Glen Research).
⁴Texas red ® (Molecular Probes, Eugene, Oreg., USA).

### Example 2—Covalent Binding of Oligonucleotides to Wells

In all experiments, 5′ amino-modified oligonucleotides were tethered in NucleoLink™ (Nalge Nunc International, Rochester, N.Y., USA) strips (eight wells/strip) by standard carbodiimide mediated condensation chemistry (Oroskar et al., “Detection of Immobilized Amplicons by ELISA-like Techniques,” Clin. Chem. 42:1547-1555 (1996), which is hereby incorporated by reference in its entirety). Oligonucleotide tethering and blocking of unreacted primer binding sites followed the microwell manufacturer's protocol (http://nunc.nalgenunc.com/resource/technical/nag/DP0063.htm), which is hereby incorporated by reference in its entirety, and which is replicated below, as disclosed by the manufacturer:

Covalent Binding of Solid Phase Primer. Make sure that the solid phase primer is phosphorylated at the 5′-end and that a linker of at least 10 T's** (Thymidine's) is added to the 5′-end of the primer. Prepare a coating mix consisting of: One ng/μl solid phase primer and 10 mM EDC (1-ethyl-3-(3-dimethylaminopropyl)-carbodiimide) in 10 mM 1-methylimidazole (1-MeIm) (pH 7.0). Add 100 μl of this freshly prepared coating mix to each well. This gives a total of 100 ng 5′-phosphorylated solid phase primer added to each well. Seal the NucleoLink Strips™ (e.g., with Nunc Sealing Tape, Cat. No. 236366). Incubate the NucleoLink Strips™ at 50° C. for 4-24 hours. Wash the empty NucleoLink™ wells three times with freshly prepared 0.4 M NaOH and 0.25% Tween 20, pre-warmed to 50° C. (it is possible to prepare the NaOH in advance and add the Tween 20 just before use). Soak for 15 minutes at 50° C. with freshly prepared 0.4 M NaOH and 0.25% Tween 20, pre-warmed to 50° C. Wash three times with freshly prepared 0.4 M NaOH and 0.25% Tween 20, pre-warmed to 50° C. Empty the strips thoroughly. To remove NaOH residues, wash the empty NucleoLink™ wells three times, soak for 5 minutes and wash three times, all with distilled water at Room Temperature (RT). Empty the NucleoLink Strips™. The coated and washed, empty, NucleoLink Strips™ can be stored at 4° C. or below in an polythene bag. The NucleoLink Strips™ should not be sealed.

Amplification. To block the wells before amplification, add to each well 200 μl of DIAPOPS (meaning: Detection of Immobilized Amplified Products in a One Phase System) buffer with 10 mg/ml BSA. Shake at RT for 1 hour). Empty the strips. No further washing is necessary, but it is important to completely empty the wells. The strips cannot be stored after this blocking step. Add PCR mix to the wells (normally 20 μl or 45 μl). The concentration of the two primers in the liquid phase should be in a ratio of 1:8 with the primer used as the solid phase primer in the lowest concentration. At the Nunc A/S Research Laboratory the concentration used is 25 pmol/reaction of the primer not used as the solid phase primer, and 25/8 pmol/reaction of the primer used as the solid phase primer. A concentration of 0.1%-0.25% Tween 20 is recommended. Add DNA template to each well (the total reaction volume has been tested with both 25 μl and 50 μl). Seal the NucleoLink Strips™ with Tape 8. Place the NucleoLink Strips™ in a thermal cycler block. Place the silicone spacer plate on the tape-sealed NucleoLink Strips™ and tighten the heated lid firmly. Program the thermal cycler with temperatures and cycling parameters specific for your system, and start the program. Remove the NucleoLink Strips™ from the thermal cycler after thermal cycling and empty the NucleoLink Strips™. The liquid phase can be stored in GeNunc™ wells (GeNunc™ 120, Cat. No. 232549), sealed with tape (Nunc Sealing Tape, Cat. No.: 236366) at 4° C. in a sealed polythene bag. Wash the empty NucleoLink Strips™ three times, soak for 5 minutes, and wash three times to denature the solid phase product, all with freshly made 0.2 M NaOH and 0.1% Tween 20 at RT. Wash the empty NucleoLink™ wells three times, soak for 5 minutes, and wash three times, all with DIAPOPS buffer at RT.

### Example 3—Optimization of Spacer Length

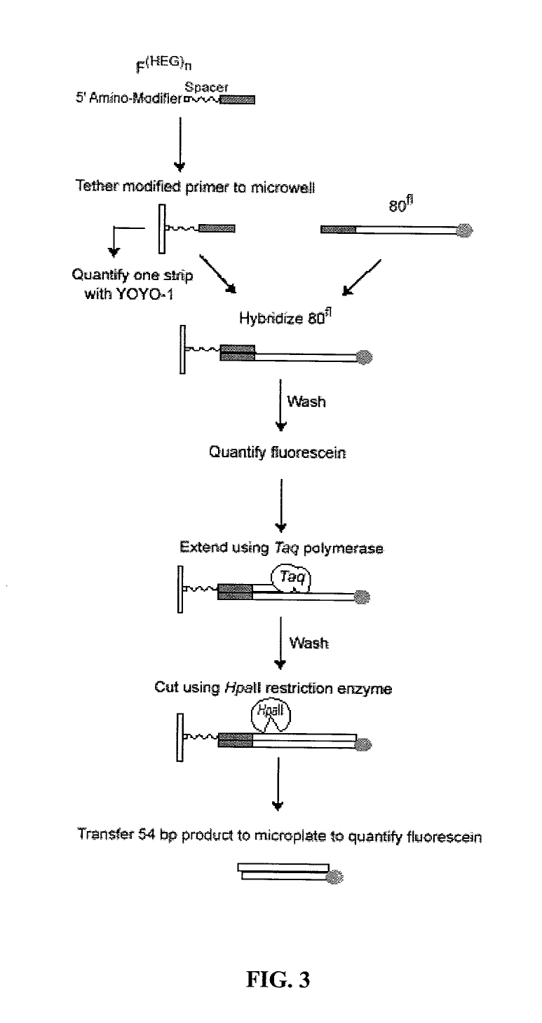

Optimal spacer length was determined as outlined in FIG. 3. Initially the F^{(HEG)}ₙ oligonucleotides with spacer lengths of 0, 5, 10 and 20 units were evaluated. Four trials (repetitions) were performed with eight 8-well strips per trial, and two wells per treatment per strip. Placement of treatments within strips was randomized. After tethering, the amount of covalently bound primer per well was determined for one strip per trial using YOYO-1 iodide (Molecular Probes, Eugene, Oreg., USA), a fluorescent dye that has a strong affinity for single-stranded DNA. Following the published protocol (Keller et al., “Use of the Fluorescent Dye YOYO-1 to Quantify Oligonucleotides Immobilized on Plastic Plates,” BioTechniques 16:1032-1034 (1994), which is hereby incorporated by reference in its entirety), fluorescence was measured with a SPECTRAFluor Plus plate reader (TECAN, Research Triangle Park, N.C., USA). After initial results were obtained, a second experiment was performed in which spacers containing from 1-8 HEG residues were evaluated. Here, each spacer length was assigned to one well per strip, but as before, eight strips were used (seven experimental strips and one strip for quantification of tethered oligonucleotide).

The 80^{fl} oligonucleotide (5 pmol) was hybridized to tethered oligonucleotides (in 100 μL 5× SSC, 1.25 M NaCl, 0.125M sodium citrate, pH 7.0) for 16 hr at 50° C. Wells were washed three times with 1× SSC at room temperature to remove unhybridized 80-mer, 100 μL of 1× SSC was added to each well, and the amount of fluorescein per well was determined using the plate reader. Tethered oligonucleotides (F^{(HEG)}ₙ) were extended in 50 μL reaction volumes containing 2.5 mM MgCl₂, 0.2 mM each dNTP, and 2.5U Taq DNA polymerase in 1× PCR buffer (Promega, Madison, Wis., USA). Reactions were incubated for 1 hr at 50° C., and wells were washed three times with 1× SSC. Restriction digests were done in 50 μL volumes with 1× One-Phor-All Buffer PLUS (Amersham Pharmacia Biotech, Piscataway, N.J., USA), 0.10 mg/mL BSA (New England BioLabs, Beverly, Mass., USA), and 1U HpaII (Life Technologies, Rockville, Md., USA). Reactions were incubated for 1 hr at 37° C. The reaction mix (40 μL) was then transferred to 96-well black plates (Corning Costar, Cambridge, Mass., USA), a 60 μL aliquot of TE pH 8.0 (10 mM Tris-HCl, 1 mM EDTA) was added, and fluorescence was measured.

Fluorescein-labeled restriction fragments were purified with Centri-Sep™ spin columns (Princeton Separations, Adelphia, N.J., USA). Samples were concentrated and approximately one-fourth of the original reaction volume was assayed on an automated DNA fragment analyzer (Applied Biosystems Model 377) using established protocols (GeneScan® Reference Guide, Applied Biosystems, Foster City, Calif., USA).

### Example 4—Quantification of Solid-phase PCR Products

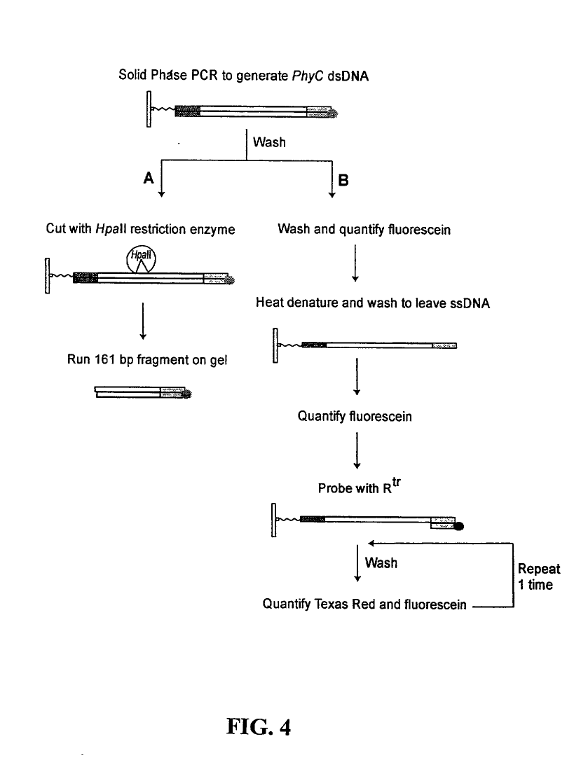

Experiments for confirmation and quantification of SP-PCR products are diagrammed in FIG. 4. Three trials were performed with four 8-well strips per trial and one treatment per strip. The presence of Taq polymerase and tethered oligonucleotides were varied in each treatment and one well per strip was reserved for quantification of tethered oligonucleotides with YOYO-1.

Total genomic DNA was extracted from Arabidopsis thaliana cv. Columbia seedlings using a standard method (5). F^{(HEG)}₅ oligonucleotides (5-unit spacers) were tethered as described. SP-PCR reaction buffers were as above, except that they contained one pmol F (unlabeled), eight pmol R^{fl} (5′-fluorescein) primers, and 25 ng Arabidopsis genomic DNA. PCR was performed using a Primus 96-plus thermocycler (MWG Biotech, Ebersberg, Germany) with the following temperature profile: 95° for 5 min, 35 cycles of 95° C. for 1 min, 55° C. for 1 min, 72° C. for 2 min, followed by a 90 min incubation at 50° C. To confirm that liquid-phase PCRs were successful, 10 μL of the amplification reactions were run on a 1% agarose gel stained with ethidium bromide. For selected wells, HpaII digests were performed, and products were sized on a DNA fragment analyzer, as before, to confirm the presence of the expected 161 bp fragment (FIG. 4A and FIG. 2).

Quantitative estimates of SP-PCR yield were made as follows. After completion of SP-PCR, wells were washed three times with 1× SSC and the amount of bound fluorescein signal was determined. Then the double-stranded DNA was denaturated by heating to 95° C. for 5 min, the solution was aspirated, the wells were washed three times with 1× SSC and fluorescein readings were obtained to measure the residual fluorescein-labeled complementary template. The tethered DNA strands were then probed with R^{tr} (Texas red-labeled) oligonucleotide (5 pmol in 50 μL 5× SSC) for 16 hr at 50° C. Wells were washed, as before, and measurements of both hybridized R^{tr} and residual fluorescein were taken. Fluorescence was determined one final time for both dyes after three additional washes with 1× SSC.

### Example 5—Comparison of 5′ HEG and dT₁₀ Spacers

F^{(HEG)}₅ and F^{(dT)}₁₀ were each tethered to all eight wells of three NucleoLink™ strips apiece and SP-PCRs were then performed as above. In addition, there were three control strips that contained all reaction components except tethered oligonucleotides. Wells were washed, probed with R^{tr}, and fluorescence measured as above. The quantity of tethered oligonucleotide was determined by YOYO-1 assay for one well per strip.

### Example 6—Statistical Analyses

Analysis of variance (ANOVA) was performed on fluorescence data using the JMP statistical software package (SAS Institute, Cary, N.C.). Box-Cox transformations were used to obtain normally distributed residuals (Box et al., “An Analysis of Transformations,” J. R. Stat. Soc. Ser. B 26:211-243 (1964), which is hereby incorporated by reference in its entirety). Linear contrast tests (Sokal et al., “Biometry,” New York, N.Y.: W. H. Freeman & Co. (1995), which is hereby incorporated by reference in its entirety) were used for planned comparisons among specific treatments. Variance components were estimated by equating observed to expected mean squares (Sokal et al., “Biometry,” New York, N.Y.: W. H. Freeman & Co. (1995), which is hereby incorporated by reference in its entirety).

### Example 7—Optimization of 5′ HEG Spacer Length

To determine the optimum length of 5′ HEG spacers on tethered primers for hybridization and extension, an experiment (FIG. 3) was designed so that hybridization could be measured independent of solid-phase extension.

Previous reports based on enzymatic assays indicated that well-to-well variability was low for the NucleoLink™ (Nalge Nunc International, Rochester, N.Y., USA) surface (Oroskar et al., “Detection of Immobilized Amplicons by ELISA-like Techniques,” Clin. Chem. 42:1547-1555 (1996), which is hereby incorporated by reference in its entirety). However, ANOVA results indicated that most of the variation in hybridization and extension experiments (65% and 83%, respectively, of the experiment-wide variance) was due to inherent differences between wells of the same strip (likely due to variability in manufacture). By contrast, there was little variability among strips (4% for hybridization and 8% for extension experiments, based on average values for eight wells per strip) and trials (31% and 9%, respectively).

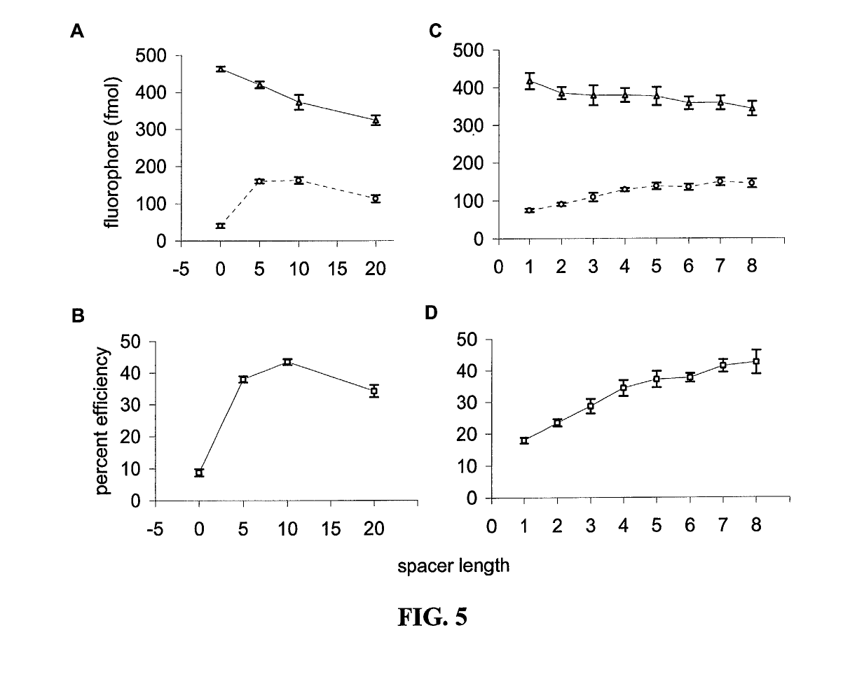

The hybridization and extension results for each spacer length are shown in FIG. 5. To establish a range for more detailed study, spacers with 0, 5, 10 and 20 HEG residues were initially evaluated. While the amount of hybridized 80-mer decreased as a function of spacer length, solid-phase primer extension increased from 0 to 5-unit spacers, was roughly equivalent for 5 and 10-unit spacers, and decreased at 20-unit spacers (FIGS. 5A and B). Thus, the comparatively inefficient extension of tethered primers without a 5′ spacer appears to be due to steric hindrance of Taq polymerase rather than to lowered hybridization efficiency. Conversely, the decline in primer extension with 20-unit spacers is likely related to decreased hybridization of the 80-mer (FIG. 5A). The optimal spacer length for efficient solid-phase extension by Taq polymerase appears to be 5-10 HEG units. When spacers between one and eight HEG units in length were evaluated, results indicated a slight decline in hybridization with increasing spacer length and an increase in the efficiency of solid-phase extension, with little difference in overall yield between 5-8 linkers (FIGS. 5C and D). Since shorter spacers are easier and less expensive to synthesize, subsequent SP-PCR experiments were performed using tethered primers with 5 units of HEG spacer.

It should be noted that other researchers have also observed decreased hybridization yields for long spacers relative to their shorter counterparts. For three different glycol spacers (propanediol, diethyleneglycol, and triethyleneglycol), Shchepinov et al., “Steric Factors Influencing Hybridization of Nucleic Acids to Oligonucleotide Arrays,” Nucleic Acids Res. 25:1155-1161 (1997), which is hereby incorporated by reference in its entirety, reported that a steady increase in duplex yield occurred with increasing spacer length up to 8-10 units but declined with further length increases, until at 30 units hybridization equaled that with no spacer at all. This effect might be due to the accumulation of negative charges with increasing spacer length (i.e. high negative charge in the spacer could repel the target DNA and result in an overall reduction in hybridization yield) (Shchepinov et al., “Steric Factors Influencing Hybridization of Nucleic Acids to Oligonucleotide Arrays,” Nucleic Acids Res. 25:1155-1161 (1997), which is hereby incorporated by reference in its entirety).

After hybridization of the 5′fluorescein-labeled 80-mer and extension of tethered primers (FIG. 3), fragments detected after digestion with HpaII should represent double-stranded extension products, since HpaII does not cut single-stranded DNA. To verify that the observed fluorescence was associated with the appropriate restriction fragment and not residual, uncut 80-mer, aliquots from selected wells were loaded on a DNA fragment analyzer. There were intense fluorescent signals around 54 bp, the size of the expected HpaII restriction fragment, and no fluorescence in the 80 bp region. Therefore, HpaII activity was either not affected by steric hindrance or the restriction enzyme excess (˜140 fold) compensated for possible steric constraints.

### Example 8—Solid-Phase PCR Verification and Quantification

The previous experiments demonstrated solid-phase extension in the presence of abundant template. To test for solid-phase extension coupled with template amplification, two experiments were performed (FIG. 4). SP-PCRs were carried out using primers that amplify a 251 bp fragment from exon I of the Arabidopsis thaliana PhyC gene (FIG. 2). The 5′aminated F^{(HEG)}₅ oligonucleotides were tethered, and PCRs were performed using a liquid-phase primer ratio of 1:8 (F:R^{fl}) to produce an excess of template strands complementary to the tethered oligonucleotide (Oroskar et al., “Detection of Immobilized Amplicons by ELISA-like Techniques,” Clin. Chem. 42:1547-1555 (1996); Rasmussen et al., “Combined Polymerase Chain Reaction-Hybridization Microplate Assay Used to Detect Bovine Leukemia Virus and Salmonella,” Clin. Chem. 40:200-205 (1994), which are hereby incorporated by reference in their entirety).

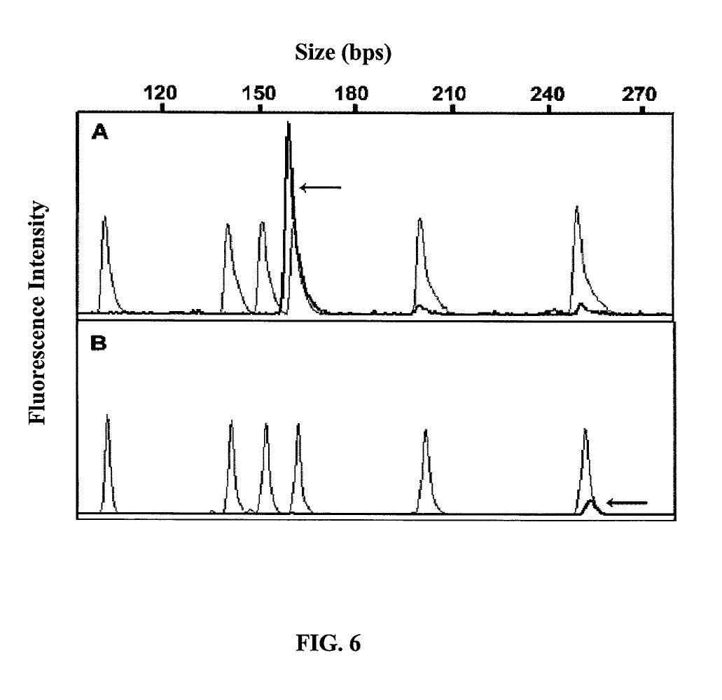

Visualization of appropriately sized (161 bp) fluorescent restriction fragments confirmed the presence of SP-PCR products in wells containing both tethered primers and Taq polymerase (FIG. 6A). Some, but not all, experimental wells (those with tethered primers and Taq) and some controls (those with Taq but without tethered primers) also contained a small amount of full-length, presumably residual single-stranded liquid-phase product (251 bp) that was not removed from wells by washing (FIG. 6B).

Fluorescence data from the experiment outlined in FIG. 4B is presented in Table 2. Statistical analyses indicates that there is a highly significant interaction term between the presence of Taq and the presence of tethered primers (p=6×10⁻⁴), and a linear contrast between the treatment having all SP-PCR components and the other three treatments revealed that the difference in fluorescence was highly significant (p=3×10⁻⁴).

TABLE 2
SP-PCR yields from various treatments expressed as mean fmol
fluorescein and Texas red.

| | | | After | After R^{tr} | | | |
|---|---|---|---|---|---|---|---|
| Treatment¹ | | After PCR | Denature | Hybridization | | After Further Washes | |
| Taq | Tether² | fluorescein³ | fluorescein | fluorescein | Texas red⁴ | fluorescein | Texas red |
| − | − | 25 (6)⁵ | 21 (6) | 6 (8) | 91 (10) | 7 (7) | 94 (6) |
| − | + | 17 (5) | 24 (5) | 4 (5) | 73 (5) | 6 (7) | 72 (5) |
| + | − | 33 (8) | 26 (5) | 2 (5) | 53 (5) | 3 (4) | 50 (5) |
| + | + | 124 (10) | 29 (7) | 21 (6) | 263 (25) | 14 (5) | 252 (23) |

¹Experiment consisted of three trials with four 8-well strips per trial and one treatment per strip.
²F^{(HEG)}₅was tethered in microwells.
³From R^{fl}-labeled PCR products and/or R^{fl} primer alone.
⁴From R^{tr} probe.
⁵2X standard error is shown in parentheses.

In this experiment (FIG. 4B), fluorescein was quantified after completion of SP-PCR and after subsequent washings and hybridizations. The fluorescein signal represented either specific binding of unincorporated R^{fl} liquid phase primers and/or fluorescein-labeled complementary PCR products to extended primers or nonspecific background. After completion of SP-PCR, approximately 100 fmol fluorescein were detected in wells containing all reaction components (Table 2). Fluorescein signal dropped to background after heat denaturation, indicating that the fluorescein-labeled complements/primers were removed from wells. The quantity of solid-phase oligonucleotides extended during PCR was estimated by hybridization to R^{tr} (Texas red-labeled probe). Although background fluorescence was relatively high in the controls, the Texas red signal from wells containing all SP-PCR components was approximately 3-fold greater. After correction for background fluorescence in the Texas red data (Table 2), it is estimated that ˜180 fmol of tethered primers was extended by SP-PCR. This is consistent with the values obtained in earlier hybridization/extension experiments where ˜160 fmol of product was detected for tethered oligonucleotides with 5 unit HEG spacers (FIG. 5A).

The Texas red background remained after three additional washes with 1× SSC at room temperature (Table 2). Among the controls, the highest background readings were in wells containing neither Taq polymerase nor tethered oligonucleotides. However, the fluorescein readings in these wells were low, even immediately following completion of temperature cycling. Since the R^{tr} probe appears to be interacting directly with the well surface, additional blocking steps, different blocking solutions, shorter hybridizations, or an alternative dye may improve the signal to noise ratio.

Based on combined YOYO-1 assays from all experiments, it is estimated that the amount of tethered primer was 780±30 fmol per well (n=72), and the density of primers on the well surface was 14 fmol (or 8×10⁹ molecules)/mm². Coupled with the SP-PCR yields obtained above, these data show that 20%, or 1 in 5, of the covalently bound primers were extended during SP-PCR. This result is a substantial improvement over estimates of SP-PCR efficiency using other approaches (e.g., 1 in 300 primers extended at an equivalent density on a glass surface in ref. 1).

### Example 9—5′ HEG vs. (dT)₁₀ Spacers

The SP-PCR yields from wells that had been tethered with F^{(dT)}₁₀ and F^{(HEG)}₅ oligonucleotides were compared. The (HEG)₅ spacer (i.e., a Spacer Phosphoramidite 18 comprising five hexaethylene glycol molecules) resulted in two fold more fluorescence than the (dT)₁₀ spacer (i.e., a polydeoxythymidine spacer comprising 10 thymidines), which in turn was only ˜150% of the background (no primer) value (Table 3). Statistical analysis indicated that there were significant differences among the blank, (dT)₁₀, and (HEG)₅ treatments (p<0.001). ANOVA results showed that the (HEG)₅ spacer resulted in significantly greater yield than the (dT)₁₀ spacer (p<0.0001) and that the (dT)₁₀ spacer had significantly higher yield than the blank (p<0.0001).

TABLE 3
Quantification of tethered primer and extended solid-phase
product with spacers of different composition expressed in mean
fmol dye¹.

| | Tethered oligo | Product extended |
|---|---|---|
| 5′ Spacer | fmol YOYO-1 | fmol Texas red |
| (HEG)₅ | 744 (81)² | 228 (8) |
| (dT)₁₀ | 713 (23) | 147 (3) |
| Control³ | 55 (7) | 52 (2) |

¹Experiment consisted of three 8-well strips per spacer with one treatment per strip. YOYO-l was quantified in one well per strip.
²2X standard error is shown in parentheses.
³Control wells contained all reactants except tethered primers.

In general, the surface density of tethered oligonucleotides, the abundance and accessibility of complementary template molecules in solution, and the accessibility of tethered primers to Taq polymerase will affect the efficiency of SP-PCR (Adessi et al., “Solid Phase DNA Amplification: Characterization of Primer Attachment and Amplification Mechanisms,” Nucleic Acids Res. 28:87e (2000) and Guo et al., “Direct Fluorescence Analysis of Genetic Polymorphisms By Hybridization With Oligonucleotide Arrays On Glass Supports,” Nucleic Acids Res. 22:5456-5465 (1994), which are hereby incorporated by reference in their entirety). Since SP-PCR products are usually detected by enzymatic reactions that are sensitive to less than one attomole of product per 20 μL volume (Kohsaka et al., “Solid-Phase Polymerase Chain Reaction,” J. Clin. Lab. Anal. 8:452-455 (1994), which is hereby incorporated by reference in its entirety), enzyme-based assays succeed if only a small proportion of tethered oligonucleotides are extended during SP-PCR, or if residual liquid-phase products remain in reaction wells. Although enzymatic detection can be done in high-throughput format, it is not quantitative and requires multiple handling steps, chemically modified probes, and expensive substrates. Here, it has been demonstrated that direct fluorescent detection of SP-PCR products is feasible in NucleoLink™ strips. Similar results are to be expected using other commercial strips or plates as long as the tethering chemistry results in 5′ immobilization of sufficient quantities of oligonucleotides on the well surface. SP-PCR yields from tethered oligonucleotides with 5′ (HEG)₅ spacers are significantly higher than yields from oligonucleotides with 5′ (dT)₁₀ spacers. The protocol of the present invention results in a 60-fold increase in extension of tethered oligonucleotides relative to reported values (Adessi et al., “Solid Phase DNA Amplification: Characterization of Primer Attachment and Amplification Mechanisms,” Nucleic Acids Res. 28:87e (2000), which is hereby incorporated by reference in its entirety). Thus, direct detection of solid-phase amplification products should now provide a simple, quantitative, cost effective means of sample analysis in a variety of molecular applications.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method for detecting a target nucleic acid molecule in a sample, said method comprising:
providing a first oligonucleotide primer coupled by a linking agent to a solid substrate, wherein said first oligonucleotide primer is complementary to at least 18 contiguous nucleic acid residues of a first strand of a target nucleic acid molecule;
contacting the first oligonucleotide primer with the sample under conditions effective to permit any of the first strand of the target nucleic acid molecule present in the sample to hybridize to the first oligonucleotide primer;
extending the first oligonucleotide primer hybridized to the first strand of the target nucleic acid molecule under conditions effective to yield a double stranded extension product coupled by the linking agent to the solid substrate, wherein the linking agent is configured to position the first oligonucleotide primer sufficiently apart from the solid substrate to permit said extending;
denaturing the extension product under conditions effective to yield an immobilized extension portion complementary to the target nucleic acid molecule;
contacting the immobilized extension portion with a detection probe, having a nucleotide sequence like that of the target nucleic acid molecule and a label, under conditions effective to permit the detection probe to hybridize specifically to the immobilized extension portion; and
detecting the label immobilized on the solid substrate, thereby indicating a presence or absence of the target nucleic acid molecule in the sample.

2. The method according to claim 1, wherein the target nucleic acid molecule is a gene locus of an organism having DNA as its genetic information.

3. The method according to claim 2, wherein the organism is selected from the group consisting of humans, animals, plants, fungi, bacteria, and viruses.

4. The method according to claim 1, wherein said method is used to detect infectious diseases caused by bacterial, viral, parasitic, and fungal infectious agents.

5. The method according to claim 4, wherein the infectious disease is caused by a bacteria selected from the group consisting of Escherichia coli, Salmonella, Shigella, Klebsiella, Pseudomonas, Listeria monocytogenes, Mycobacterium tuberculosis, Mycobacterium avium-intracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasma, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, and Actinomycetes.

6. The method according to claim 4, wherein the infectious disease is caused by a fungal infectious agent selected from the group consisting of Cryptococcus neoformans, Blastomyces dermatitidis, Histoplasma capsulatum, Coccidioides immitis, Paracoccicioides brasiliensis, Candida albicans, Aspergillus fumigautus, Phycomycetes, Sporothrix schenckii, Chromomycosis, and Maduromycosis.

7. The method according to claim 4, wherein the infectious disease is caused by a viral infectious agent selected from the group consisting of human immunodeficiency virus, human T-cell lymphocytotrophic virus, hepatitis viruses, Epstein-Barr Virus, cytomegalovirus, human papillomaviruses, orthomyxo viruses, paramyxo viruses, adenoviruses, corona viruses, rhabdo viruses, polio viruses, toga viruses, bunya viruses, arena viruses, rubella viruses, and reo viruses.

8. The method according to claim 4, wherein the infectious disease is caused by a parasitic infectious agent selected from the group consisting of Plasmodium falciparum, Plasmodium malaria, Plasmodium vivax, Plasmodium ovale, Onchoverva volvulus, Leishmania, Trypanosoma spp., Schistosoma spp., Entamoeba histolytica, Cryptosporidum, Giardia spp., Trichimonas spp., Balatidium coli, Wuchereria bancrofti, Toxoplasma spp., Enterobius vermicularis, Ascaris lumbricoides, Trichuris trichiura, Dracunculus medinesis, trematodes, Diphyllobothrium latum, Taenia spp., Pneumocystis carinii, and Necator americanis.

9. The method according to claim 1, wherein said method is used to detect genetic diseases.

10. The method according to claim 9, wherein the genetic disease has a known nucleotide sequence and is selected from the group consisting of 21 hydroxylase deficiency, cystic fibrosis, Fragile X Syndrome, Turner Syndrome, Duchenne Muscular Dystrophy, Down Syndrome, heart disease, single gene diseases, HLA typing, phenylketonuria, sickle cell anemia, Tay-Sachs Syndrome, thalassemia, Klinefelter's Syndrome, Huntington's Disease, autoimmune diseases, lipidosis, obesity defects, hemophilia, inborn errors in metabolism, and diabetes.

11. The method according to claim 1, wherein said method is used to detect cancer having a known nucleotide sequence and involving oncogenes, tumor suppressor genes, or genes involved in DNA amplification, replication, recombination, or repair.

12. The method according to claim 11, wherein the cancer is associated with a gene selected from the group consisting of BRCA1 gene, p53 gene, Familial polyposis coli, Her2/Neu amplification, Bcr/Ab1, K-ras gene, human papillomavirus Types 16 and 18, leukemia, colon cancer, breast cancer, lung cancer, prostate cancer, brain tumors, central nervous system tumors, bladder tumors, melanomas, liver cancer, osteosarcoma and other bone cancers, testicular and ovarian carcinomas, ENT tumors, and loss of heterozygosity.

13. The method according to claim 1, wherein said method is used for environmental monitoring, forensics, and food and feed industry monitoring.

14. The method according to claim 1, wherein the linking agent does not include a nucleic acid.

15. The method according to claim 1, wherein the linking agent has a length of about 5 to about 500 Ångstroms.

16. The method according to claim 15, wherein the linking agent has a length of about 25 to 250 Ångstroms.

17. The method according to claim 1, wherein said coupling of the first oligonucleotide primer with the linking agent is by a covalent bond.

18. The method according to claim 1, wherein said linking agent is generated by a 5′-Amino Modifier C6 spacer.

19. The method according to claim 18, wherein said 5′-Amino Modifier C6 spacer comprises the following chemical structure:

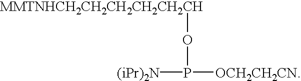

20. The method according to claim 1, wherein said linking agent comprises a polyethylene glycol spacer.

21. The method according to claim 20, wherein said polyethylene glycol spacer is selected from the group consisting of triethylene glycol spacers, hexaethylene glycol spacers, and heptaethylene glycol spacers.

22. The method according to claim 21, wherein said hexaethylene glycol spacer is generated through the use of a Spacer Phosphoramidite 18 having the following structure:

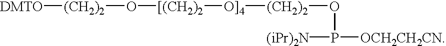

23. The method according to claim 22, wherein said Spacer Phosphoramidite 18 is used to introduce between about 1 to 20 hexaethylene glycol molecules into said hexaethylene glycol spacer.

24. The method according to claim 1, wherein said linking agent is generated using a 5′-Amino Modifier C6 spacer coupled to a polyethylene glycol spacer.

25. The method according to claim 24, wherein said 5′-Amino Modifier C6 is of the formula:

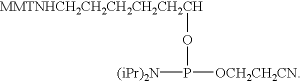

26. The method according to claim 25, wherein said hexaethylene glycol spacer is generated through the use of Spacer Phosphoramidite 18 having the following formula:

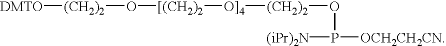

27. The method according to claim 26, wherein said Spacer Phosphoramidite 18 is used to generate between about 1 to 20 hexaethylene glycol molecules.

28. The method according to claim 1, wherein the solid substrate is in a form selected from the group consisting of wells, microtiter plates, slides, discs, columns, beads, membranes, films, and composites thereof.

29. The method according to claim 28, wherein the solid substrate is functionalized with olefin, amino, hydroxyl, silanol, aldehyde, keto, halo, acyl halide, or carboxyl groups to permit attachment of the first oligonucleotide primer to the solid substrate.

30. The method according to claim 29, wherein the solid substrate is functionalized with an amino group by reaction with an amine compound selected from the group consisting of 3-aminopropyl triethoxysilane, 3-aminopropylmethyldiethoxysilane, 3-aminopropyl dimethylethoxysilane, 3-aminopropyl trimethoxysilane, N-(2-aminoethyl)-3-aminopropylmethyl dimethoxysilane, N-(2-aminoethyl-3-aminopropyl) trimethoxysilane, aminophenyl trimethoxysilane, 4-aminobutyldimethyl methoxysilane, 4-aminobutyl triethoxysilane, aminoethylaminomethylphenethyl trimethoxysilane, and mixtures thereof.

31. The method according to claim 29, wherein the solid substrate is functionalized with an olefin-containing silane.

32. The method according to claim 31, wherein the olefin-containing silane is selected from the group consisting of 3-(trimethoxysilyl)propyl methacrylate, N-[3-(trimethoxysilyl)propyl]-N′-(4-vinylbenzyl)ethylenediamine, triethoxyvinylsilane, triethylvinylsilane, vinyltrichlorosilane, vinyltrimethoxysilane, vinyltrimethylsilane, and mixtures thereof.

33. The method according to claim 29, wherein the solid substrate is functionalized with a silanol polymerized with an olefin-containing monomer.

34. The method according to claim 33, wherein the olefin-containing monomer contains a functional group.

35. The method according to claim 33, wherein the olefin-containing monomer is selected from the group consisting of acrylic acid, methacrylic acid, vinylacetic acid, 4-vinylbenzoic acid, itaconic acid, allyl amine, allylethylamine, 4-aminostyrene, 2-aminoethyl methacrylate, acryloyl chloride, methacryloyl chloride, chlorostyrene, dichlorostyrene, 4-hydroxystyrene, hydroxymethylstyrene, vinylbenzyl alcohol, allyl alcohol, 2-hydroxyethyl methacrylate, poly(ethylene glycol) methacrylate, and mixtures thereof.

36. The method according to claim 28, wherein the solid substrate is a polymer produced from a monomer selected from the group consisting of acrylic acid, methacrylic acid, vinylacetic acid, 4-vinylbenzoic acid, itaconic acid, allyl amine, allylethylamine, 4-aminostyrene, 2-aminoethyl methacrylate, acryloyl chloride, methacryloyl chloride, chlorostyrene, dischlorostyrene, 4-hydroxystyrene, hydroxymethyl styrene, vinylbenzyl alcohol, allyl alcohol, 2-hydroxyethyl methacrylate, poly(ethylene glycol) methacrylate, and mixtures thereof, together with a monomer selected from the group consisting of acrylic acid, acrylamide, methacrylic acid, vinylacetic acid, 4-vinylbenzoic acid, itaconic acid, allyl amine, allylethylamine, 4-aminostyrene, 2-aminoethyl methacrylate, acryloyl chloride, methacryloyl chloride, chlorostyrene, dichlorostyrene, 4-hydroxystyrene, hydroxymethyl styrene, vinylbenzyl alcohol, allyl alcohol, 2-hydroxyethyl methacrylate, poly(ethylene glycol) methacrylate, methyl acrylate, methyl methacrylate, ethyl acrylate, ethyl methacrylate, styrene, 1-vinylimidazole, 2-vinylpyridine, 4-vinylpyridine, divinylbenzene, ethylene glycol dimethacrylate, N,N′-methylenediacrylamide, N,N′-phenylenediacrylamide, 3,5-bis(acryloylamido) benzoic acid, pentaerythritol triacrylate, trimethylolpropane trimethacrylate, pentaerytrithol tetraacrylate, trimethylolpropane ethoxylate (14/3 EO/OH) triacrylate, trimethylolpropane ethoxylate (7/3 EO/OH) triacrylate, trimethylolpropane propoxylate (1 PO/OH) triacrylate, trimethylolpropane propoxylate (2 PO/OH) triacrylate, and mixtures thereof.

37. The method according to claim 28, wherein the solid substrate is a microwell suitable for use in quantitative assays that employ direct fluorescence detection.

38. The method according to claim 1, wherein said extending is carried out in an extension reaction mixture comprising dATP, dCTP, dTTP, dGTP, dITP, dUTP, and a polymerizing agent.

39. The method according to claim 38, wherein the polymerizing agent is selected from the group consisting of Thermus aquaticus DNA polymerase, Thermus thermophilus DNA polymerase, E. coli DNA polymerase, T4 DNA polymerase, and Pyrococcus DNA polymerase.

40. The method according to claim 1, wherein the detection probe has a hybridization temperature of 20-85° C.

41. The method according to claim 1, wherein the label is selected from the group consisting of chromophores, fluorescent dyes, enzymes, antigens, heavy metals, magnetic probes, dyes, phosphorescent groups, radioactive materials, chemiluminescent moieties, electrochemical detecting moieties, and specific mass tags.

42. The method according to claim 41, wherein the label is a fluorescent dye selected from the group consisting of fluorescein, rhodamine, Texas Red, allophycocyanin, propidium iodide, Cy5, Cascade Blue, Dansyl, dialklyamino-coumarin, eosin, erythrosin, isosulfan blue, malachite green, Oregon green, pyrene, rhodamine green, rhodamine red, rhodol green, and derivatives of these fluorescent dyes.


SEQUENCE LISTING (6 sequences)

SEQ ID NO: 1 - DNA, 20 bp, Arabidopsis thaliana
gcctttttatgcgattctgc

SEQ ID NO: 2 - DNA, 30 bp, Arabidopsis thaliana
ttttttttttgcctttttatgcgattctgc

SEQ ID NO: 3 - DNA, 80 bp, Arabidopsis thaliana
caggcacctcatcaggactcacaggatccaaatctataacaagaccttcctcaatccggtgcagaatcgcataaaaaggc

SEQ ID NO: 4 - DNA, 20 bp, Arabidopsis thaliana
gcctttttatgcgattctgc

SEQ ID NO: 5 - DNA, 20 bp, Arabidopsis thaliana
cgggtaggagtaccttgaat

SEQ ID NO: 6 - DNA, 20 bp, Arabidopsis thaliana
cgggtaggagtaccttgaat